(19) United States
(12) Patent Application Publication
(10) Pub. No.: US 2017/0086798 A1
(43) Pub. Date: Mar. 30, 2017

(54) OPTIMAL UTILIZATION OF BANDWIDTH BETWEEN ULTRASOUND PROBE AND DISPLAY UNIT

(71) Applicant: General Electric Company, Schenectady, NY (US)

(72) Inventors: Steinar Bjaerum, Horten (NO); Trond Kleveland, Horten (NO); Trond Kierulf, Horten (NO); Per Arne Linnerud, Horten (NO)

(21) Appl. No.: 14/871,191

(22) Filed: Sep. 30, 2015

(51) Int. Cl. A61B 8/00; A61B 8/08

(52) U.S. Cl. CPC A61B 8/565; A61B 8/4444; A61B 8/461; A61B 8/5207; A61B 8/483

(57) ABSTRACT

Various embodiments include systems and methods for optimizing utilization of bandwidth between ultrasound probes and display units. Data transfer limits for a connection between an ultrasound probe and a corresponding display-and-control unit may be determined. Based on the data transfer limits, one or more functions in the ultrasound probe may be controlled. The one or more functions relate to acquiring of ultrasound image data via the ultrasound probe, processing of the acquired ultrasound image data, and/or communication of the acquired ultrasound image data. Further, the controlling is adapted to reduce amount of data acquired and/or transferred, between the ultrasound probe and the display-and-control unit, during ultrasound imaging, to meet the data transfer limits for the connection.

### CLAIMS OF PRIORITY

[Not Applicable]

### CROSS-REFERENCE TO RELATED APPLICATIONS/INCORPORATION BY REFERENCE

[Not Applicable]

### FIELD OF THE INVENTION

Certain embodiments of the invention relate to ultrasound imaging. More specifically, certain embodiments of the invention relate to methods and systems for optimizing utilization of bandwidth between ultrasound probes and display units.

### BACKGROUND OF THE INVENTION

Ultrasound imaging is a medical imaging technique for imaging organs and soft tissues in a human body. Ultrasound imaging uses real time, non-invasive high frequency sound waves to produce ultrasound images. These ultrasound images may be two-dimensional (2D), three-dimensional (3D), and/or four-dimensional (4D) images.

Equipment used in ultrasound imaging may comprise multiple individual components. For example, an ultrasound imaging machine may comprise a portable component (e.g., ultrasound probe) that is used in capturing the images, and a main component (e.g., display unit) that is used in presenting (e.g., displaying) the images to the machine operator. The main component may also be configured to provide additional/remaining functions associated with ultrasound imaging—e.g., processing the images, interfacing with the operator (e.g., to obtain user input, including commands, settings, preference, etc.), and the like. Such multi-component arrangements may necessitate use of connections for exchanging data (control data, images, etc.) between the components. The connections may be wired (e.g., cords, cable, and the like) and/or wireless (e.g., using radio frequency signals configured based on particular wireless interface/protocol, such as WiFi, etc.).

Ultrasound imaging may entail transfer of large data. This may be the case, for example, with 3D or 4D imaging where high-quality real-time images are captured by the ultrasound probes, and are then transferred to the display units. In some instances, however, there may be limitations on data transfers. For example, in multi-component arrangements described above, bandwidth limitations associated with the connections used between the different component (e.g., between the ultrasound probe and the display unit) may exist. This may be particularly the case with wireless connections.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY OF THE INVENTION

A system and/or method is provided for optimal utilization of bandwidth between ultrasound probe and display unit, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a block diagram illustrating an example ultrasound system that may be used in ultrasound imaging, which may also support optimizing utilization of bandwidth during data transfers, in accordance with various embodiments of the invention.

FIG. 2 is a block diagram illustrating an example ultrasound system that is operable to provide optimal utilization of bandwidth of wireless connection between ultrasound probe and display unit, in accordance with an example embodiment of the invention.

FIG. 3 is a flow chart illustrating example steps that may be performed when optimizing utilization of bandwidth for data transfers during ultrasound imaging, in accordance with an embodiment of the invention.

### DETAILED DESCRIPTION OF THE INVENTION

Certain embodiments of the invention may be found in methods and systems for optimizing utilization of bandwidth during ultrasound imaging, such as bandwidth between ultrasound probes and display units. For example, aspects of the present invention have the technical effect of optimizing utilization of a connection between an ultrasound probe and a corresponding control/display unit in the same ultrasound system, by controlling one or more functions in the ultrasound probe based on data transfer limits (e.g., based on available bandwidth) of the connection. In this regard, the functions may relate to acquiring of ultrasound image data via the ultrasound probe, processing of the acquired ultrasound image data, and/or communication of the acquired ultrasound image data. Controlling the functions in the ultrasound probe may be adapted such that it may have technical effect of reducing amount of data acquired and/or transferred, between the ultrasound probe and the display-and-control unit, during ultrasound imaging, to meet the data transfer limits for the connection.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments of the present invention. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an embodiment,” “one embodiment,” “a representative embodiment,” “an example embodiment,” “various embodiments,” “certain embodiments,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

In addition, as used herein, the phrase “pixel” also includes embodiments of the present invention where the data is represented by a “voxel.” Thus, both the terms “pixel” and “voxel” may be used interchangeably throughout this document.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode and/or sub-modes of CF such as TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the invention, such as single or multi-core: CPU, Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams.” Also, forming of images may be performed using channel combinations that may originate from more than one transmit event (e.g., synthetic aperture techniques).

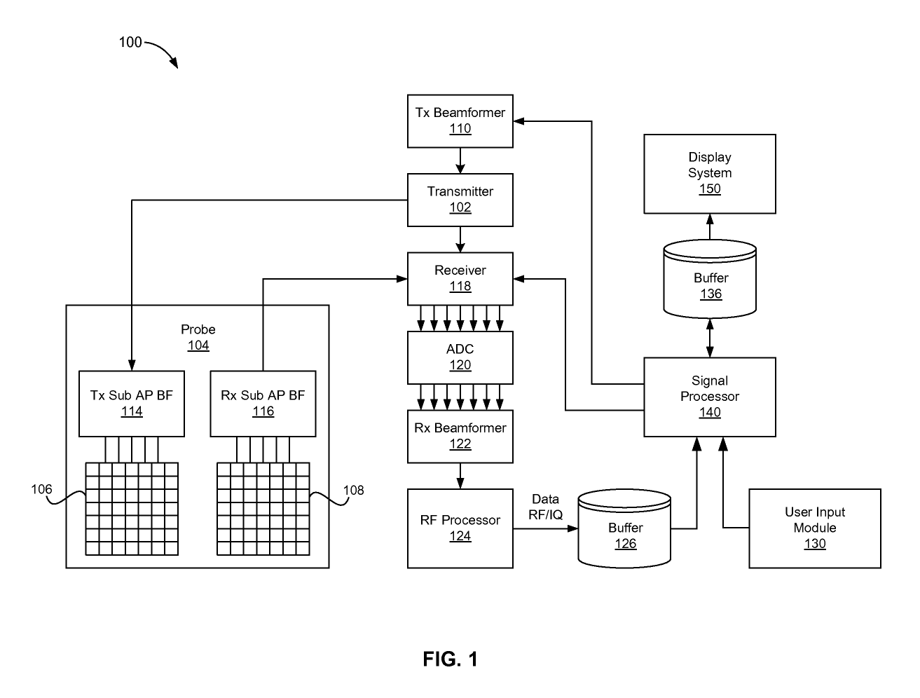

In various embodiments, ultrasound processing, including visualization enhancement, to form images may be performed, for example, in software, firmware, hardware, or a combination thereof. One implementation of an ultrasound system in accordance with various embodiments is illustrated in FIG. 1.

FIG. 1 is a block diagram illustrating an example ultrasound system that is operable to provide optimal utilization of bandwidth between ultrasound probe and display unit, in accordance with an embodiment of the invention. Shown in FIG. 1 is an ultrasound system 100.

The ultrasound system 100 comprises, for example, a transmitter 102, an ultrasound probe 104, a transmit beamformer 110, a receiver 118, a receive beamformer 122, a RF processor 124, a RF/IQ buffer 126, a user input module 130, a signal processor 140, an image buffer 136, and a display system 150.

The transmitter 102 may comprise suitable circuitry that may be operable to drive an ultrasound probe 104. The transmitter 102 and the ultrasound probe 104 may be implemented and/or configured for one dimensional (1D), two-dimensional (2D), three-dimensional (3D), and/or four-dimensional (4D) ultrasound scanning. In this regard, ultrasound probe 104 may comprise a one dimensional (1D, 1.25D, 1.5D or 1.75D) array or a two dimensional (2D) array of piezoelectric elements. For example, as shown in FIG. 1, the ultrasound probe 104 may comprise a group of transmit transducer elements 106 and a group of receive transducer elements 108, that normally constitute the same elements. The transmitter 102 may be driven by the transmit beamformer 110.

The transmit beamformer 110 may comprise suitable circuitry that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). In this regard, the group of transmit transducer elements 106 can be activated to transmit ultrasonic signals. The ultrasonic signals may comprise, for example, pulse sequences that are fired repeatedly at a pulse repetition frequency (PRF), which may typically be in the kilohertz range. The pulse sequences may be focused at the same transmit focal position with the same transmit characteristics. A series of transmit firings focused at the same transmit focal position may be referred to as a “packet.”

The transmitted ultrasonic signals may be back-scattered from structures in the object of interest, like tissue, to produce echoes. The echoes are received by the receive transducer elements 108. The group of receive transducer elements 108 in the ultrasound probe 104 may be operable to convert the received echoes into analog signals, undergo sub-aperture beamforming by a receive sub-aperture beamformer 116 and are then communicated to the receiver 118.

The receiver 118 may comprise suitable circuitry that may be operable to receive and demodulate the signals from the probe transducer elements or receive sub-aperture beamformer 116. The demodulated analog signals may be communicated to one or more of the plurality of A/D converters (ADCs) 120.

Each plurality of A/D converters 120 may comprise suitable circuitry that may be operable to convert analog signals to corresponding digital signals. In this regard, the plurality of A/D converters 120 may be configured to convert demodulated analog signals from the receiver 118 to corresponding digital signals. The plurality of A/D converters 120 are disposed between the receiver 118 and the receive beamformer 122.

Notwithstanding, the invention is not limited in this regard. Accordingly, in some embodiments of the invention, the plurality of A/D converters 120 may be integrated within the receiver 118.

The receive beamformer 122 may comprise suitable circuitry that may be operable to perform digital beamforming processing to, for example, sum the delayed channel signals received from the plurality of A/D converters 120 and output a beam summed signal. The resulting processed information may be converted to corresponding RF signals. The corresponding output RF signals that are output from the receive beamformer 122 may be communicated to the RF processor 124. In accordance with some embodiments of the invention, the receiver 118, the plurality of A/D converters 120, and the beamformer 122 may be integrated into a single beamformer.

The RF processor 124 may comprise suitable circuitry that may be operable to demodulate the RF signals. In some instances, the RF processor 124 may comprise a complex demodulator (not shown) that is operable to demodulate the RF signals to form In-phase and quadrature (IQ) data pairs (e.g., B-mode data pairs) which may be representative of the corresponding echo signals. The RF (or IQ) signal data may then be communicated to an RF/IQ buffer 126.

The RF/IQ buffer 126 may comprise suitable circuitry that may be operable to provide temporary storage of output of the RF processor 124—e.g., the RF (or IQ) signal data, which is generated by the RF processor 124.

The user input module 130 may comprise suitable circuitry that may be operable to enable obtaining or providing input to the ultrasound system 100, for use in operations thereof. For example, the user input module 130 may be used to input patient data, surgical instrument data, scan parameters, settings, configuration parameters, change scan mode, and the like. In an example embodiment of the invention, the user input module 130 may be operable to configure, manage and/or control operation of one or more components and/or modules in the ultrasound system 100. In this regard, the user input module 130 may be operable to configure, manage and/or control operation of transmitter 102, the ultrasound probe 104, the transmit beamformer 110, the receiver 118, the receive beamformer 122, the RF processor 124, the RF/IQ buffer 126, the user input module 130, the signal processor 140, the image buffer 136, and/or the display system 150.

The signal processor 140 may comprise suitable circuitry that may be operable to process the ultrasound scan data (e.g., the RF and/or IQ signal data) and/or to generate corresponding ultrasound images, such as for presentation on the display system 150. The signal processor 140 is operable to perform one or more processing operations according to a plurality of selectable ultrasound modalities on the acquired ultrasound scan data. In some instances, the signal processor 140 may be operable to perform compounding, motion tracking, and/or speckle tracking. Acquired ultrasound scan data may be processed in real-time—e.g., during a B-mode scanning session, as the B-mode echo signals are received. Additionally or alternatively, the ultrasound scan data may be stored temporarily in the RF/IQ buffer 126 during a scanning session and processed in less than real-time in a live or off-line operation.

While the ultrasound probe 104 is shown as comprising only the transmit transducer elements 106, the transmit sub-aperture beamformer 114, the receive transducer elements 108, and the receive sub-aperture beamformer 116, the invention is not so limited to such arrangement. Thus, in various embodiments of the invention other elements (e.g., one or more of the other components of the ultrasound system 100, such as the transmit beamformer 110, the receiver 118, the A/D converters 120, the receive beamformer 122, the RF processor 124, the RF/IQ buffer 126, and the signal processor 140) may be incorporated into the ultrasound probe 104.

In operation, the ultrasound system 100 may be used in generating ultrasonic images, including two-dimensional (2D), three-dimensional (3D), and/or four-dimensional (4D) images. In this regard, the ultrasound system 100 may be operable to continuously acquire ultrasound scan data at a particular frame rate, which may be suitable for the imaging situation in question. For example, frame rates may range from 20-70 but may be lower or higher. The acquired ultrasound scan data may be displayed on the display system 150 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several seconds' worth of frames of ultrasound scan data. The frames of ultrasound scan data are stored in a manner to facilitate retrieval thereof according to its order or time of acquisition. The image buffer 136 may be embodied as any known data storage medium.

In some instances, the ultrasound system 100 may be configured to support grayscale and color based operations. For example, the signal processor 140 may be operable to perform grayscale B-mode processing and/or color processing. The grayscale B-mode processing may comprise processing B-mode RF signal data or IQ data pairs. For example, the grayscale B-mode processing may enable forming an envelope of the beam-summed receive signal by computing the quantity (I²+Q²)^{1/2}. The envelope can undergo additional B-mode processing, such as logarithmic compression to form the display data. The display data may be converted to X-Y format for video display. The scan-converted frames can be mapped to grayscale for display. The B-mode frames that are provided to the image buffer 136 and/or the display system 150. The color processing may comprise processing color based RF signal data or IQ data pairs to form frames to overlay on B-mode frames that are provided to the image buffer 136 and/or the display system 150. The grayscale and/or color processing may be adaptively adjusted based on user input—e.g., a selection from the user input module 130, for example, for enhance of grayscale and/or color of particular area.

In some instances, ultrasound systems, such as the ultrasound system 100, may comprise multiple separate physical elements, each of which comprising suitable components for performing various particular functions and/or operations associated with ultrasound imaging. For example, the ultrasound system 100 may comprise a portable element (e.g., the ultrasound probe 104, as well as, in some instances, some of the other functional components described above) that is used in capturing the images, and a display/control element (or display/control unit), which is used in providing such functions as presenting (e.g., displaying) the images to the operator, processing the images (for display), supporting user interfacing (e.g., to obtain user input, including commands, settings, preference, etc.), and the like. As noted above, in such arrangements comprising multiple separate physical elements connections may be used for exchanging data (control data, images, etc.) between the different elements. In this regard, the connections between the different elements may be wired (e.g., cords, cable, and the like) and/or wireless (e.g., using radio frequency signals configured based on particular wireless interface/protocol, such as WiFi, etc.). Ultrasound imaging may entail transfer of large data, however. This may be particularly the case with, for example, 3D or 4D ultrasound imaging, with color-based ultrasound imaging, etc. where high-quality real-time images (or data corresponding thereto) may be captured or generated by one element (e.g., by the ultrasound probes), and may then need to be transferred to other elements (e.g., the display/control unit). In some instances, there may be limitations on data transfers, however. For example, bandwidth limitations associated with the connections used between the different element (e.g., between the ultrasound probe and the display/control unit) may exist. This may be particularly the case with wireless connections.

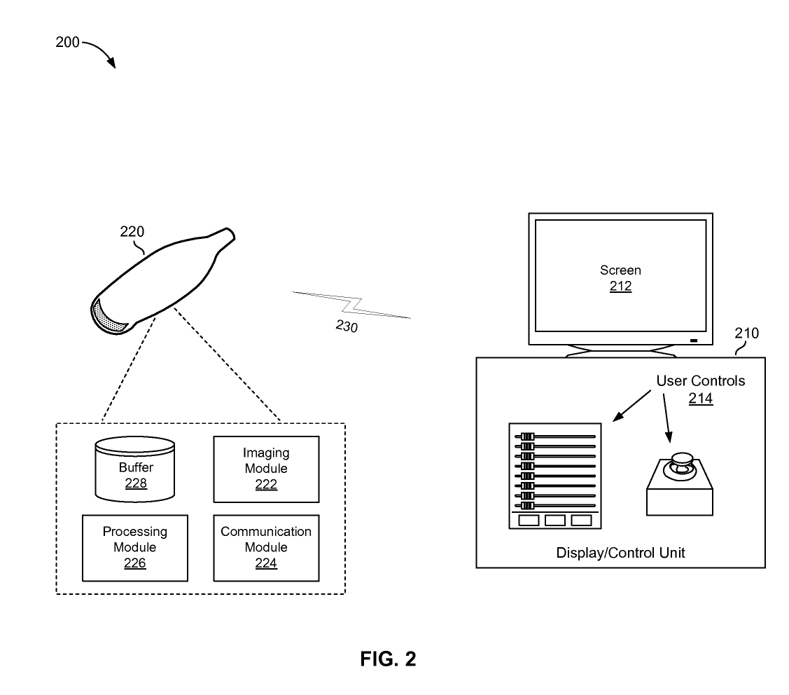

Accordingly, in various embodiments according to the present invention, use of connection between different elements of ultrasound systems (particularly with respect to bandwidth utilization in these connections) may be optimized, particularly in adaptive and dynamic manner during ultrasound imaging. In this regard, optimizing bandwidth use may comprise applying one or more optimization measures to reduce the data transferred between the different elements and/or to reduce the overall data transfer rate (e.g., by spreading out data transfer over time, such that to avoid large bursts of data). An example ultrasound system that is configured to provide bandwidth optimization is described in more detail with respect to FIG. 2.

The optimization measures may comprise or be directed to controlling the amount of data generated or transferred (e.g., to reduce its size), adaptive management of the connections (e.g., to reduce total and instantaneous data transfers, such as by queuing data till bandwidth becomes available, communicating portions of data based on available bandwidth, adjusting communication settings or parameter, such as modulation or compression, to increase data transfer efficiency, etc.), adaptive management the ultrasound imaging (e.g., to reduce amount of data generated or transferred, by adjusting ultrasound parameters and/or characteristics, for example, such as temporal and/or spatial resolution, selective imaging of particular regions, etc.). These measures may be adaptively selected and/or configured based on available bandwidth, which may be continually assessed and/or monitored.

Use of the optimization measures may not pertain to and/or be based on the total amount of data in an ultrasound data set; rather, the focus and/or determining factor is real-time transfer data rate available during the acquisition and/or transfer of ultrasound data set (from the ultrasound probe). In this regard, the real-time transfer data rate is limited by available links or connections (wired and/or wireless), between the ultrasound probe and the rest of the ultrasound system, and limitations with these links or connections (e.g., available bandwidth). Thus, the optimization measures may be used to ensure that the imaging and/or data acquired and/or transferred based thereon conform to real-time transfer data rate limitations. This may be done in different ways, each of which having certain advantages and/or disadvantages, which may be assessed during the selection and/or configuration of optimization measures.

For example, the optimization measures may be directed to reducing the amount of acquired data, such as by reducing the acquisition data rate so that the data rate after (simple) processing fits into the available transfer data rate (e.g., based on available bandwidth). Reducing the acquisition data rate may comprise reducing the spatial resolution (number of spatial sample points) and/or reducing the temporal resolution (number of images per second). The acquired data reduction approach has the advantage of simple processing. However, it may result in degraded image quality and/or framerate, which may be limited by real-time transfer data rate available at any given point. In other words, the data set available for review, analysis, reporting, archiving, etc. may be limited (and may be substantially reduced) whenever the real-time transfer data rate is limited.

The optimization measures may also be directed to reducing the data that has already been acquired, such as when processing the data for transfer. Thus, the data may be acquired using the most optimal acquisition criteria—e.g., using high real-time acquisition data rate. Then, when the data is transferred, it may be processed (if necessary) to reduce its size. The processing may comprise, for example, applying compression (e.g., lossy compression) to the data to meet the requirements imposed by the real-time transfer data rate. This approach (data reduction by post-acquisition processing) results in better image quality compared to the previous approach; it may, however, require significant additional processing and consequently high power consumption as well (also possibly leading to heating and battery time issues). Another issue may be the need to include added components in the ultrasound probe to handle the process, which may result in a bigger and less ergonomic ultrasound probe. The post-acquisition processing of data (particularly compression) may also lead to sub-optimal image quality. Further, the processing/compression may result in reduced flexibility in what type of processing and image analysis that may be done with the data received by the ultrasound system's main unit (where display of ultrasound images, as well as any additional processing needed to generate and/or configure the images, is done).

The optimization measures may also be directed to buffering the data, after the data is already acquired. Again, the ultrasound data may be acquired using the most optimal acquisition criteria possible—e.g., using high real-time acquisition data rate. The complete raw data set may then be buffered (if necessary) in the ultrasound probe. During real-time mode (real-time scanning), the data may be reduced, such as by decimation (spatial and/or temporal) or/and compression (lossless or lossy), in order to conform to available real-time transfer data rate of the links/connections. In this regard, it is important to distinguish between reduction in acquired data by reducing acquisition data rate, such as by reducing spatial resolution and/or temporal resolution (as done in the first approach, above), and the reduction done after acquisition, as done here, by increasing the decimation or/and compression when generating images from the raw data set stored in the data buffer in the ultrasound probe. The reduced acquisition data rate results in reduced raw data set in first scenario, whereas a full data set is acquired (and buffered) in the second case, and that data set (or copy thereof) then is only reduced for transfer.

The resulting sub-optimal real-time image quality may be configured to be similar to the image quality the prior two approaches described above. When the system ultrasound system's operator select a particular other mode (e.g., freeze mode, review mode, analysis mode, reporting mode, storage mode, etc.), the complete or missing parts of the raw data set may be transferred from the buffer in the ultrasound probe to the ultrasound system's main unit. Thus, the complete raw data set may be made available and/or transferred to the ultrasound system's main unit only when needed.

This approach (buffering of full data set) results in no loss in image quality for certain purposes or use scenarios (e.g., review, analysis, reporting, storage, etc.). Also, since the raw (full) data is available there would be no compromises for processing and measurements/analysis in ultrasound system's main unit. Further, less processing may be required compared to previous approach (post-acquisition processing), but at the cost of some degradation in real-time image quality.

The advantages and disadvantages of each of the approaches may be assessed and/or evaluated during selection and/or configuration of optimization measures that are applied. Further, rather than simply select only one of the three approaches, a combination of two or all three of the approaches may be possible. For instance, in some instances (e.g., where the transfer data rate may be extremely low) it may be necessary to apply acquired data reduction (e.g., reducing acquisition data rate) as well as measures corresponding to one or both of the other approaches (post-acquisition processing and buffering). The selection of optimization measures between the three approaches (or of any combinations thereof) may be done automatically and dynamically. In this regard, the selection may be adapted to the available real-time transfer data rate of the link.

In an example use scenario of the buffering-based approach, when scanning the patient, the ultrasound system's operator may perceive sub-optimal image quality. The sub-optimal image quality may be caused by decimation of the ultrasound data set (e.g., spatially and/or temporally). The ultrasound system's operator may then decide to review a cineloop of the current view and may provide a user input (e.g., by interacting with the user interface/user input module 130) accordingly. The cineloop may start looping on the display, and the image quality may gradually improve until the entire data set is transferred from the ultrasound probe.

In example use scenario of combined buffering and post-processing based approach, during real-time scanning, the raw data set may be buffered in the ultrasound probe, and at the same time processing may be applied to the data in the ultrasound probe to reduce the size of data transferred to the ultrasound system's main unit (e.g., generating a compressed video stream, such as MPEG, based on the data). The ultrasound system's operator may then decide to do some quantitative analysis of the current color flow data set and interacts with the user interface accordingly. The raw data set may be made available to the ultrasound system's main unit allowing full flexibility when doing image adjustments and clinical measurements. This is very similar to the JPEG+RAW datasets available from digital cameras. The RAW image allows more advanced post processing than the JPEG image.

FIG. 2 is a block diagram illustrating an example ultrasound system that is operable to provide optimal utilization of bandwidth of wireless connection between ultrasound probe and display/control unit 210, in accordance with an example embodiment of the invention. Shown in FIG. 2 is ultrasound system 200.

The ultrasound system 200 may be substantially similar to the ultrasound system 100, and as such may comprise generally similar components as described with respect to the ultrasound system 100 of FIG. 1. The ultrasound system 200 may be configured to, however, optimize use of connections (particularly bandwidth utilization) between different physical elements of the system may be optimized. In this regard, the different physical elements may be located in close proximity of each other, and may communicate with one another via local (e.g., wireless, such as WiFi) connections.

As shown in FIG. 2, the ultrasound system 200 may comprise a portable and movable ultrasound probe 220 and a display/control unit 210. The ultrasound probe 220 may be used in generating and/or capturing ultrasound images (or data corresponding thereto), such as by being moved over a patient's body (or part thereof). The display/control unit 210 may be used in displaying ultrasound images (e.g., via a screen 212). Further, the display/control unit 210 may support user interactions (e.g., via user controls 214), such as to allow controlling of the ultrasound imaging. The user interactions may comprise user input or commands controlling display of ultrasound images, selecting settings, specifying user preferences, providing feedback as to quality of imaging, etc.

In operation, the ultrasound system 200 may be used in ultrasound imaging, such as to generate and present (e.g., display) ultrasound images, including 2D, 3D, and/or 4D ultrasound images, and/or to support user input in conjunction therewith, substantially as described with respect to FIG. 1. Additionally, however, the ultrasound system 200 may be operable to optimize connection utilization (particularly with respect to bandwidth utilization in the connection(s)) between the different elements of the ultrasound system 200 (e.g., between the ultrasound probe 220 and the display/control unit 210), and to do so particularly in adaptive and dynamic manner during ultrasound imaging. For example, optimizing bandwidth use may comprise applying one or more optimization measures to optimize (e.g., reduce) the total transferred data and/or the data transfer rate between the ultrasound probe 220 and the display/control unit 210.

As noted above, this may include measures applied to the data being transferred itself (to reduce its size), measures applied to the use of connections and/or communications via the connections (e.g., adjusting type of connections, modifying communication related parameters, use of data queuing, use of data segmentation, etc.), measures applied to the ultrasound functions and/or related parameters (e.g., adjusting temporal and/or spatial resolution, dynamic temporal and/or spatial decimation or compression to adapt to available bandwidth, with subsequent reconstruction of full data set when additional bandwidth is available, etc.), selective imaging of particular regions (e.g., varying quality in images), etc.

The ultrasound probe 220 may be configured or be operable to support or enable implementation of these optimization measures. In this regard, the ultrasound probe 200 may comprise components that may be configured to provide functions or operations pertinent to the implementation of these measures. These may include components that are already incorporated into the ultrasound probe 220 (to facilitate or support functions related to the operation of the ultrasound probe 220 during ultrasound imaging), but may also comprise components that are dedicated for supporting connection (bandwidth) optimization. For example, in an example embodiment shown in FIG. 2, the ultrasound probe 220 may comprise an imaging module 222, a communication module 224, a processing module 226, and a buffer 228.

The imaging module 222 may comprise suitable circuitry that may be operable to perform the ultrasound imaging—that is capturing data corresponding to ultrasound imaging and performing at least some of the initial processing of the data in the course of generating the corresponding ultrasound images. For example, the imaging module 222 may comprise or may correspond to, at least in part, the transmit sub-aperture beamformer 114 and the receive sub-aperture beamformer 116, and may in some instance further comprise or implement at least some of the functionality of the transmitter 102, the transmit beamformer 110, the receiver 118, the plurality of A/D converters 120, and the receive beamformer 122. In some instances, the imaging module 222 may be configured to support optimizing connection (e.g., bandwidth) utilization during operation of the ultrasound system 200.

The communication module 224 may comprise suitable circuitry that may be operable to perform and/or support communication related functions or operations. For example, the communication module 224 may be configured to setup connections (e.g., wireless connection, such as WiFi connections), manage setup connections (e.g., monitoring connection condition, and modify the connections based on the monitoring—including adjusting connection parameters, tearing connections down (re)setting up connection(s), etc.), handle reception and/or transmission of signals over setup connections (which may include performing at least some of the processing needed to embed or extract data from the signals), etc. In some instances, the communication module 224 may be configured to perform communication-related functions or operations pertaining to and/or in support of optimizing connection (e.g., bandwidth) utilization in the ultrasound system 200.

The processing module 226 may comprise suitable circuitry that may be operable to perform various processing functions or operations. For example, the processing module 226 may be configured to control operations of the ultrasound probe 220 (and its various components), and/or to perform processing pertaining to ultrasound imaging. Further, in some instances, the processing module 226 may be configured to perform processing functions or operations pertaining to optimizing connection (e.g., bandwidth) utilization during operation of the ultrasound system 200.

The buffer 228 may comprise suitable circuitry that may be operable to provide temporary storage of data during operations of the ultrasound probe 220. For example, the buffer 228 may buffer data during operations via one or more of the imaging module 222, the processing module 226, and the communication module 224. In some instances, the buffer 228 may be configured to buffer data in support of connection (e.g., bandwidth) use optimization operations in the ultrasound system 200.

As noted above, optimizing connection (or bandwidth thereof) utilization may comprise use of one or more measures or techniques to ensure that data corresponding to captured ultrasound images is transferred in an optimal manner—that is within the communication constraints (e.g., available bandwidth) of the available connections between the elements (e.g., between the ultrasound probe 220 and the display/control unit 210), while maintaining good (e.g., or acceptable) image quality, such as in accordance with set quality criteria (e.g., based on user input or pre-defined settings). In this regard, in some instances, it may not be possible to transfer all the acquired ultrasound data in particular communication environments—e.g., via wireless connections, in real time in a noisy environment that does not support high data transfer rates. Thus, data and/or data transfers may be controlled and/or managed to utilize available bandwidth in the best manner that may still ensure the good (e.g., or acceptable) image quality as much as possible. In this regard, optimizing connection (or bandwidth thereof) utilization may comprise reducing the amount of data generated and/or transferred during ultrasound imaging. This may also be done by reducing amount of data captured or generated, such as by adjusting imaging functions (via the imaging module 222) and/or data processing data functions (via one or more of the imaging module 222, the processing module 226, and the communication module 224), and/or reducing the amount of data transferred or reducing the data transfer rates, such as by adjusting communication functions (via the communication module 224).

For example, the amount of data captured or generated may be reduced—that as the amount of data as initially acquired and/or after subsequent processing thereof. This may be done, for example, by scanning only a subset of the lines/vectors, or by reducing the number of frames per second. Adaptive variable line spacing may also be used. For example, rather than scanning the whole image uniformly, the imaging operations may be configured (e.g., via the imaging module 222) to scan differentially—e.g., to scan such that only particular regions in the image may be shown more clearly (e.g., corresponding to certain organs/objects, such as the heart walls), with the rest of the image being subject to more widely spaced scan lines.

In some instances, the amount of data transferred may be reduced, such as by be automatically transmitted lower-quality real-time ultrasound images (or data corresponding thereto). This may be done adaptively—that is based on limitations and/or availability of connection (e.g., bandwidth thereof). The ultrasound probe 220 may acquire and buffer high quality ultrasound data, but may only transmit only a particular subset of the data to the display/control unit 210 in a low bandwidth environment. The subset may be selected adaptively—e.g., that is to permit the user to have a lower-quality real-time image for basic ultrasound operations, finding the correct scan plane, etc.

Also, some of the data may not be transferred, with that “missing” data being buffered in the ultrasound probe (e.g., in the buffer 228). The missing (raw) data, which may be necessary for building or assembling the complete high quality data set, may be transferred when possible—e.g., based on bandwidth availability, or entering the particular mode (the “freeze mode.”)

In some instances, the ultrasound system 200 (or the ultrasound probe 220) may transition, such as based on determination of communication (e.g., bandwidth) limitation that does not permit transfer of all data corresponding to the captured images, to a particular mode of operation (e.g., “freeze mode”, thus ceasing capturing of additional images).

In some instances, all of the data acquired for a particular preceding period of time (e.g., prior 5 or 10 seconds), or in some instance portion of that data (e.g., the “missing” data), which may be buffered (e.g., via the buffer 228), may then be transferred (adaptively, via the communication module 224, based on real-time assessment of the connection conditions), to allow for a higher quality image for archiving and analysis. The data transfer rate may be adaptively adjusted (continually), such that data transfers may be done at a higher rate where possible (e.g., when the network environment supports high data transfer rates).

In some instances, data type may be adjusted to ensure compliance with available bandwidth. For example, for video data rather than transfer conventional video, compressed video format may be applied to would-be-transferred data. When more detailed data (or velocity raw data for Doppler modes) is needed, such as for measurement, analysis, or for reporting, the ultrasound probe 220 may transmit high quality raw data, which may be buffered (e.g., in the buffer 228), to the display/control unit 210. In this regard, when bandwidth to transmit becomes available (e.g., after transmitting of previously queued data, or when we are in an environment supporting high transfer rates), the raw data (beams/vectors) may be transferred. For example, rolling buffer of particular length (e.g., 10 sec.) may be used.

In some instances, different types of data may be handled in different ways at the same time, to best reduce amount of data while achieve best possible ultrasound imaging quality. For example, where transferred data comprises video data (e.g., where the processing to generate the images to be displayed is performed in the ultrasound probe 220), the video data may encoded to different more efficient format (e.g., to MPEG or another video format) before transferring, to reduce amount of data transferred. Wherein non-video data (e.g., beam space data) is being transferred—that is where the processing to generate the final images is performed outside the probe, such as in the display/control unit 210, other measures may be applied to optimize the data and/or transfer thereof. For example, the beam space data may be reduced by reducing number of samples (e.g., by reducing spatial and/or temporal sampling) and/or transferring every other beam, a reduced framerate may be used, lossless or lossy data compression may be applied on data before transmission, etc.

In some instances, certain ultrasound related parameters or characteristics may be adjusted based on data transfer limitations (e.g., available bandwidth), such as to reduce amount of data captured and/or processed for transmission. For example, one or both of the spatial or temporal resolution may be adjusted—e.g., by the user, via the user controls 214 of the display/control unit 210, with the imaging module 222 being adjusted accordingly to effectuated these changes.

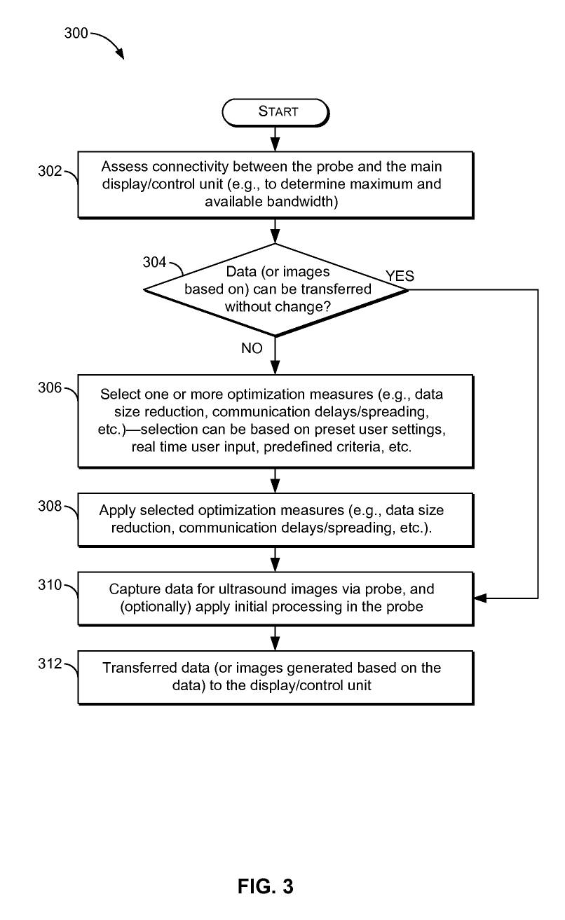

FIG. 3 is a flow chart illustrating example steps that may be performed when optimizing utilization of bandwidth for data transfers during ultrasound imaging, in accordance with an embodiment of the invention. Shown in FIG. 3 is a flow chart 300, which comprises a plurality of example steps, corresponding to an example method.

The technical effect of the method corresponding to flow chart 300 is providing adaptive visualization in volumetric ultrasound images by an ultrasound system (e.g., the ultrasound system 200, shown in FIG. 2). For example, the example steps of the method corresponding to flow chart 300 may be executed and/or performed by the various components of the ultrasound system 200, such as the imaging module 222, the processing module 224, the processing module 226, and the buffer 228.

It should be understood, however, that certain embodiments of the present invention may omit one or more of the steps, and/or perform the steps in a different order than the order listed, and/or combine certain of the steps discussed below. For example, some steps may not be performed in certain embodiments of the present invention. As a further example, certain steps may be performed in a different temporal order, including simultaneously, than listed below.

In step 302, after a start step (in which an ultrasound system may be, for example, initialized and/or configured for ultrasound imaging), connectivity conditions between one element of an ultrasound system (e.g., an ultrasound probe, such as the ultrasound probe 220 of the ultrasound system 200 of FIG. 2) and a second element of the ultrasound system (e.g., a main unit, such as the display/control unit 210 of the ultrasound system 200 of FIG. 2) may be assessed.

In step 304, it may be determined whether ultrasound images (or data corresponding thereto) acquired using current imaging settings may be transferred under current connectivity conditions. In instances where it may be determined that ultrasound images (or data corresponding thereto) captured under current imaging conditions may be transferred, the process may jump to step 310; otherwise the process may proceed to step 306.

In step 306, one or more optimization measures (e.g., ultrasound imaging adjustments, data size reduction, communication delays/spreading, etc. as described above) may be selected for application to the ultrasound imaging and/or data corresponding thereto. The selection can be based on, for example, user settings or preferences, real time user input, predefined criteria, etc.

In step 308, the selected optimization measures (e.g., data size, reduction, communication delays/spreading, etc.) may be applied to the ultrasound imaging and/or to ultrasound images (or acquired data corresponding thereto). This may include, for example, adjusting how data is acquired, adjusting acquired data (or portion thereof), modifying ultrasound parameters or functions, buffering portions of the date, etc.

In step 310, data corresponding to a sequence of ultrasound images may be obtained, such as using the ultrasound probe.

In step 312, ultrasound image data (or images generated based on the data) may be transferred to the main unit, such as to facilitate displaying the ultrasound images.

As utilized herein the term “circuitry” refers to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or.” As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “example” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments of the invention may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for optimizing connection (e.g., bandwidth thereof) utilization between portable ultrasound probes and corresponding display units in ultrasound systems.

Accordingly, the present invention may be realized in hardware, software, or a combination of hardware and software. The present invention may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

The present invention may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method, comprising:
determining data transfer limits for a connection between an ultrasound probe and a corresponding display-and-control unit; and
controlling, based on the data transfer limits, one or more functions in the ultrasound probe; wherein:
the one or more functions relate to acquiring of ultrasound image data via the ultrasound probe, processing of data, and/or communication of data to the display-and-control unit; and
the controlling is adapted to reduce amount of data acquired in the ultrasound probe and/or transferred between the ultrasound probe and the display-and-control unit, during ultrasound imaging, to meet the data transfer limits for the connection.

2. The method of claim 1, wherein the connection comprises a wireless connection.

3. The method of claim 1, wherein communicated data comprises one or more of: at least a portion of the acquired ultrasound image data, at least a portion of a buffered ultrasound image data, and at least a portion of processed ultrasound image data.

4. The method of claim 1, wherein controlling the one or more functions in the ultrasound probe comprises:
acquiring full ultrasound image data set;
selecting at least a portion of the full ultrasound image data set for transfer to the display-and-control unit;
buffering data corresponding to full ultrasound image data set or non-transferred portion of the ultrasound image data set; and
when additional bandwidth is available; communicating the buffered data to the display-and-control unit.

5. The method of claim 1, wherein controlling the one or more functions in the ultrasound probe comprises adjusting ultrasound imaging related functions and/or parameters to reduce acquired ultrasound image data.

6. The method of claim 5, wherein the ultrasound imaging related functions and/or parameters comprise temporal resolution and/or spatial resolution.

7. The method of claim 5, comprising adjusting ultrasound imaging related functions and/or parameters based on varying imaging criteria for different regions in corresponding ultrasound images.

8. The method of claim 1, wherein controlling the one or more functions in the ultrasound probe comprises applying compression to data transferred between the ultrasound probe and the display-and-control unit.

9. The method of claim 1, wherein controlling the one or more functions in the ultrasound probe comprises applying temporal and/or spatial decimation to data transferred between the ultrasound probe and the display-and-control unit.

10. A system, comprising:
a display-and-control unit that comprises a display for displaying ultrasound images; and
an ultrasound probe that is operable to acquire ultrasound image data, the ultrasound probe comprising one or more circuits operable to:
determine data transfer limits for a connection between the ultrasound probe and the display-and-control unit; and
controlling, based on the data transfer limits, one or more functions in the ultrasound probe; wherein:
the one or more functions relate to acquiring of ultrasound image data via the ultrasound probe, processing of data, and/or communication of data to the display-and-control unit; and
the controlling is adapted to reduce amount of data acquired in the ultrasound probe and/or transferred between the ultrasound probe and the display-and-control unit, during ultrasound imaging, to meet the data transfer limits for the connection.

11. The system of claim 10, wherein the connection comprises a wireless connection.

12. The system of claim 10, wherein communicated data comprises one or more of: at least a portion of the acquired ultrasound image data, at least a portion of a buffered ultrasound image data, and at least a portion of processed ultrasound image data.

13. The system of claim 10, wherein controlling the one or more functions in the ultrasound probe comprises:
acquiring full ultrasound image data set;
selecting at least a portion of the full ultrasound image data set for transfer to the display-and-control unit;
buffering data corresponding to full ultrasound image data set or non-transferred portion of the ultrasound image data set; and
when additional bandwidth is available; communicating the buffered data to the display-and-control unit.

14. The system of claim 10, wherein controlling the one or more functions in the ultrasound probe comprises adjusting ultrasound imaging related functions and/or parameters to reduced acquired ultrasound image data.

15. The system of claim 14, wherein the ultrasound imaging related functions and/or parameters comprise temporal resolution and/or spatial resolution.

16. The system of claim 14, wherein the one or more circuits are operable to adjust ultrasound imaging related functions and/or parameters based on varying imaging criteria for different regions in corresponding ultrasound images.

17. The system of claim 10, wherein controlling the one or more functions in the ultrasound probe comprises applying compression to data transferred between the ultrasound probe and the display-and-control unit.

18. The system of claim 10, wherein controlling of the one or more functions in the ultrasound probe comprises dynamically applying temporal and/or spatial decimation to data transferred between the ultrasound probe and the display-and-control unit.

19. A non-transitory computer readable medium having stored thereon, a computer program having at least one code section, the at least one code section being executable by a machine for causing the machine to perform steps comprising:
determining data transfer limits for a connection between an ultrasound probe and a corresponding display-and-control unit; and
controlling, based on the data transfer limits, one or more functions in the ultrasound probe; wherein:
the one or more functions relate to acquiring of ultrasound image data via the ultrasound probe, processing of data, and/or communication of data to the display-and-control unit; and
the controlling is adapted to reduce amount of data acquired in the ultrasound probe and/or transferred between the ultrasound probe and the display-and-control unit, during ultrasound imaging, to meet the data transfer limits for the connection.

20. The non-transitory computer readable medium of claim 19, wherein the controlling of the one or more functions in the ultrasound probe comprises:
acquiring full ultrasound image data set;
selecting at least a portion of the full ultrasound image data set for transfer to the display-and-control unit;
buffering data corresponding to full ultrasound image data set or non-transferred portion of the ultrasound image data set; and
when additional bandwidth is available; communicating the buffered data to the display-and-control unit.
